(19) United States
(12) Patent Application Publication
(10) Pub. No.: US 2025/0195595 A1
(43) Pub. Date: Jun. 19, 2025

(54) TOPICAL FORMULATION FOR WOUND HEALING

(71) Applicant: Oneness Biotech Co., Ltd., Taipei City (TW)

(72) Inventors: Kung-Ming Lu, Taipei City (TW); Hui-Ling Cheng, Taipei City (TW); Chou-Hsiung Chen, Taipei City (TW)

(21) Appl. No.: 18/982,369

(22) Filed: Dec. 16, 2024

Related U.S. Application Data
(60) Provisional application No. 63/610,742, filed on Dec. 15, 2023.

(51) Int. Cl. A61K 36/23; A61K 9/00; A61K 9/06; A61K 31/728; A61K 36/53; A61K 38/17; A61K 38/39

(52) U.S. Cl. CPC A61K 36/23; A61K 9/0014; A61K 9/06; A61K 31/728; A61K 36/53; A61K 38/1748; A61K 38/39

(57) ABSTRACT

A topical formulation comprising a Plectranthus amboinicus (PA) extract, a Centella asiatica (CA) extract, a moisture-retaining polymeric biomaterial, and a pharmaceutically effective excipient. The present disclosure also relates to the use of the topical formulation for promoting wound healing.

### CROSS REFERENCE TO RELATED APPLICATIONS

This application claims the benefit of the filing date of U.S. Provisional Application No. 63/610,742, filed Dec. 15, 2023, the entire contents of which is incorporated by reference herein.

### BACKGROUND OF THE INVENTION

Plectranthus amboinicus (also known previously or alternatively as Coleus amboinicus Lour., Coleus aromaticus Benth., Coleus aromaticus auct., Plectranthus aromaticus Roxb., Plectranthus aromaticus Benth., and Plectranthus amboinicus (Lour.) Spreng.), is a perennial medicinal herb of the Lamiaceae (also known as Labiatae) family native to Southern and Eastern Africa. Plectranthus amboinicus is also known as patchouli, Cuban oregano, Indian borage, Indian mint, Mexican mint, Mexican oregano, country borage, and Spanish thyme.

Centella asiatica (also known previously or alternatively as Centella asiatica Urban, Centella asiatica (L.) Urban, Hydrocotyle asiatica L., and Trisanthus cochinchinensis Lour.) is a perennial medicinal plant of the family Mackinlayaceae or subfamily Mackinlayoideae of the Apiaceae (also known as Umbelliferae) family native to Asia, Africa, and South America. Centella asiatica is also known as European water-marvel, gotu kola, Kola, pennywort, Indian pennywort, marsh pennywort, pennyweed, Indian ginseng, Horse-hoof grass, Pegaga, Mandookaparni, Tiger herbal, Spadeleaf, or Tono. Extracts of Centella asiatica generally comprise two major compounds: asiaticoside and madecassoside.

### SUMMARY OF THE INVENTION

The present disclosure is based, at least in part, on the development of botanic extract-containing topical formulations that exhibit superior moisture-retaining capacity, which contributes to, at least in part, the asserted therapeutic efficacy of the topical formulations (e.g., promoting wound healing). In some instances, the topical formulations provided herein may comprise botanic extracts (e.g., Plectranthus amboinicus (PA) extract, and/or a Centella asiatica (CA) extract), and a moisture-retaining polymeric biomaterial as described herein.

Accordingly, provided herein is a topical formulation, comprising: (i) a PA extract; (ii) a CA extract; (iii) a moisture-retaining polymeric biomaterial; and optionally (iv) a pharmaceutically effective excipient.

In some embodiments, the topical formulation may comprise (i) about 0.1-20% (w/w) of the PA extract; (ii) about 0.1-40% (w/w) of the CA extract; and/or (iii) about 0.1-90% (w/w) (e.g., about 0.1-70%) of the moisture-retaining polymeric biomaterial.

In some embodiments, the moisture-retaining polymeric biomaterial may comprise collagen, karatin, hyaluronate acid or a salt thereof (e.g., sodium hyaluronate), or a combination thereof. In some specific examples, the moisture-retaining polymeric biomaterial is collagen from fish, porcine, or bovine.

In some embodiments, the topical formulation may be in the form of a cream, a gel, an ointment, or a sheet dressing.

In one embodiment, the topical formulation is a cream formulation, which may comprise the pharmaceutically effective excipient together with the PA extract, the CA extract, and the moisture-retaining polymeric biomaterial such as collagen. The pharmaceutically effective excipient may comprise an oil base, an emulsifying agent, and water. In some instances, the oil base comprises fatty acid ester, aliphatic higher alcohol, paraffin oil, lanolin oil, silicone oil, medium-chain oil, and/or silicone-based polymer. By way of non-limiting examples, the oil base may comprise petrolatum and/or dimethicone. In some instances, the emulsifying agent comprises an anionic surfactant, a cationic surfactant, a zwitterionic surfactant, and/or a non-ionic surfactant. By way of non-limiting examples, the emulsifying agent may comprise a polysorbate compound, a sorbitan compound, or a combination thereof.

In any of the cream formulations described herein, the pharmaceutically effective excipient may further comprise one or more of the following: (a) a skin conditioning agent; (b) an emollient; (c) a stiffening agent; (d) a humectant; and (e) a preservative.

In specific examples, the topical formulation as provided herein, in cream format, may comprise the PA extract in an amount of about 0.1-10% by weight, the CA extract in an amount of about 0.1-24% by weight; and/or the collagen in an amount of about 0.1-20% by weight. Such a cream formulation may further comprise white petrolatum and water.

In another embodiment, the topical formulation is a gel formulation, wherein the pharmaceutically effective excipient comprises a gelling agent and water. In some instances, the gelling agent comprises a cellulose derivative, a carbomer, or a combination thereof. By way of non-limiting examples, the gelling agent may be carboxymethyl cellulose. In any of the gel formulations described herein, the pharmaceutically effective excipient may further comprise an emollient and/or a preservative.

In still another embodiment, the topical formulation is an ointment formulation, wherein the pharmaceutically effective excipient comprises an ointment base. In some instances, the ointment base comprises a polyethylene glycol, an oil base, or a combination thereof. By way of non-limiting examples, the ointment base may comprise petrolatum and/or dimethicone. In any of the ointment formulations described herein, the pharmaceutically effective excipient may further comprise a preservative. In some embodiments, (e.g., for multiple uses after opening), the topical formulations may contain one or more antimicrobial preservatives to prevent any microorganism growth. Alternatively, the topical formulation (e.g., for single use packaging), may be free of any preservatives.

In specific examples, the ointment formulation provided herein may comprise the PA extract in an amount of about 0.1-20% by weight; the CA extract in an amount of about 0.1-40% by weight; and/or the collagen in an amount of about 0.1-70% by weight. Such an ointment formulation may further comprise white petrolatum.

In yet another embodiment, the topical formulation is a sheet dressing, wherein the pharmaceutically effective excipient comprises a dressing. In some instances, the dressing may be a bandage, a membrane, a synthetic or biological hydrogel, a hydrocolloid, a film, a foam, a gauze, a dermal patch, an adhesive tape, a skin substitute, a paste, a powder, or a fiber. In any of the sheet dressings described herein, the pharmaceutically effective excipient may further comprise an antioxidant, a growth factor, and/or an extracellular matrix.

In specific examples, the sheet dressing provided herein may comprise the PA extract in an amount of about 24% by weight, the CA extract in an amount of about 8-15% by weight, and/or collagen in an amount of about 80-90% by weight.

In another aspect, the instant disclosure provides a method for wound healing, comprising administering the topical formulation disclosed herein to a wound site of a subject in need thereof. In one embodiment, the topical formulation can be administered at least once per day, for example, twice daily.

In one example, the subject is a human patient having an acute or open wound, for example, abrasion, incision, laceration, puncture, or avulsion. In another example, the subject is a human patient having a chronic wound, which may be a surgical wound, a traumatic wound, a pressure ulcer, a venous ulcer, a diabetic ulcer, or a wound caused by a carcinoma, burn, atopic dermatitis, bedsore.

Also within the scope of the present disclosure are (i) a topical formulation as described herein for use in promoting wound healing, and (ii) uses of the topical formulation provided herein in manufacturing a medicament for use in promoting wound healing.

The details of one or more embodiments of the invention are set forth in the description below. Other features or advantages of the present invention will be apparent from the following drawings and detailed description of several embodiments, and also from the appended claims.

### DETAILED DESCRIPTION OF THE INVENTION

The present disclose is based, at least in part, on the development of a topical formulation comprising a Plectranthus amboinicus (PA) extract, a Centella asiatica (CA) extract, a moisture-retaining polymeric biomaterial, and a pharmaceutically effective excipient. Performance test showed the topical formulation provided herein possessed exceptional moisture retention capacity (e.g., 97.1%˜100.0% in cream or ointment format), which is superior to the commercial topical formulation products (without moisture-retaining polymeric biomaterials such as collagen) for treating skin conditions, for example, ALHYDRAN® (94.5%) and BIAFINE® (93.8%). Such superior moisture-retaining capacity of the topical formulations provided herein suggests that they can be used to alleviate skin damage (e.g., promoting wound healing) via, e.g., retention of moisture and/or suppression of inflammation with high efficacy.

Accordingly, provided herein are topical formulations that comprise a PA extract, a CA extract, a moisture-retaining polymeric biomaterial, and a pharmaceutically effective excipient, as well as methods of using the same for promoting wound healing in a subject in need of the treatment.

### I. Topical Formulations

The instant disclosure herein provides a topical formulation for alleviating skin damages, for example, promoting wound healing, the topical formulation may comprise a PA extract, a CA extract, a moisture-retaining polymeric biomaterial, and optionally a pharmaceutically effective excipient.

### A. Botanic Extracts

Any of the topical formulations may comprise botanic extracts such as PA and/or CA extracts. The topical formulation may comprise about 0.1-5%, 0.5-3%, 1-2%, 1-3%, 2-10%, 5-10%, 5-15%, 10-15%, 15-20%, 10-20%, 20-25%, 15-30%, 20-30%, 10-30%, or 25-30% (w/w) of the botanic extract. The concentration of the botanic extract (or other components disclosed herein) in the topical formulation described herein refers to the percentage of the total weight of the botanic extract in dry form of the total weight of the topical formulation as a whole.

In some instances, the topical formulation described herein may comprise the PA extract in an amount ranging from about 0.1% to about 20% (w/w), for example, about 0.1% to about 15%, about 0.1% to about 10%, about 0.1% to about 5%, or about 0.1% to about 2% by weight. In some examples, the PA extract may be in an amount of about 0.1-1% (e.g., about 0.1-05% such as about 0.25% w/w).

Alternatively or in addition, the topical formulation described herein may comprise the CA extract in an amount ranging from about 0.1% to about 40% (w/w), for example, about 0.1% to about 35%, about 0.1% to about 30%, about 0.1% to about 25%, about 0.1% to about 20%, about 0.1% to about 15%, about 0.1% to about 15%, about 0.1% to about 10%, or about 0.1% to about 5% by weight. In some examples, the CA extract may be in an amount of about 0.1-3% (e.g., about 0.1-2% or about 0.1-1.5% such as about 1% w/w).

As used herein, the term “about” intends to a strict numerical boundary to the specified parameters (including both the upper and lower limits). A skilled person in the art would have understood the meaning of “about” in association with a specific context. In some instances, the term “about” refers to a particular value+/−5% (e.g., +/−3% or +/−2%).

In some instances, the ratio between the Plectranthus amboinicus extract and the Centella asiatica extract may be about 1:10 to 10:1. For example, the ratio between the Plectranthus amboinicus extract and the Centella asiatica extract in the topical formulation may be about 1:10 to 1:1, about 1:1 to 10:1, about 1:10 to 1:5, about 1:5 to 1:1, about 10:1 to 5:1, about 5:1 to 1:1, about 1:10 to 1:4, about 1:4 to 1:1, about 10:1 to 4:1, or about 4:1 to 1:1.

### (a) Plectranthus Amboinicus (PA) Extract

A Plectranthus amboinicus (PA) extract refers to an extract obtained from Plectranthus amboinicus plant using one or more suitable solvents. In some instances, at least one of the solvents used for preparing the extract has a polarity index lower than 7 (e.g., less than 5). See, e.g., US20180015133, the relevant disclosures of which are incorporated by reference herein for the subject matter and purpose referenced herein. As used herein, a solvent refers to a substance or a mixture of substances that dissolves another to form a solution. A PA extract as described herein may be prepared using one solvent. The solvent used in each extracting step for preparing the extracts described herein (including both PA and CA extracts) may be a single solvent. Alternatively, it can be a mixture of two or more solvents.

The PA extract described herein may comprise terpenoids (e.g., monoterpenoids, diterpenoids, triterpenoids, and/or sesquiterpenoids), flavonoids, phenolics, essential oils, or a combination thereof. The PA extract may be present in the topical formation at a concentration of about 0.1-5% (w/w). For example, the topical formulation may comprise about 0.1-0.5%, 0.5-1%, 1-1.5%, 1.5-2%, 2-2.5%, 2.5-3%, 3-3.5%, 3.5-4%, 4-4.5%, or 4.5-5% (w/w) of the PA extract. Alternatively, the topical formulation may comprise about 0.1-1%, 0.1-2%, 0.1-3%, 0.1-4%, or 0.1-5% percent (w/w) of the PA extract. In a specific embodiment, the topical formulation comprises about 0.1-1% or 0.1-0.5% of the PA extract.

The PA extract described herein may be prepared by extracting a whole PA plant or a part thereof (e.g., an aerial part) with one or more suitable solvents to produce a solution and then drying the solution to produce the PA extract. Since a PA extract comprises flavonoids, terpenoids (e.g., monoterpenoids, diterpenoids, triterpenoids, and/or sesquiterpenoids), phenolics, or essential oils, which are non-polar molecules, at least one of the extracting solvents may have a relatively low polarity (e.g., having a polarity index lower than 7) to facilitate dissolution of the non-polar molecules. “Extracting” can be performed by either contacting the PA material directly with a suitable solvent or by eluting active components of PA from resins on which the active components are attached.

In some examples, a solvent having a polarity index lower than 7 can be used for extracting the active components from Plectranthus amboinicus to produce the Plectranthus amboinicus extract. Such a solvent can be ethyl acetate, methyl acetate, propanol, butanol, or chloroform. Alternatively, the solvent can be a mixture of one or more solvents having different polarity index. Examples include, but are not limited to, a mixture of ethanol and ethyl acetate, ethyl acetate and butanol, ethanol and propanol, methyl acetate and butanol.

In some embodiments, the PA extract may be prepared by a process involving the use of one solvent, such as a solvent having a polarity index lower than 7 (e.g., <6.5, <6.0; <5.5, <about 5.0, <4.9, <4.8, <4.7, <4.6, or <4.5). Examples include, but are not limited to, methanol, ethanol, acetone, ethyl acetate, butanol, dichloromethane, or a combination thereof.

PA materials, which can be a whole PA or a part thereof (e.g., an above-ground part such as leaves), can be prepared by routine methodology. The PA material can be a fresh plant or a part thereof. Alternatively, the PA material can be in dried form. The PA can optionally be dried to form powders, which can be used as the PA material for preparing the PA extract.

Any of the PA materials as described herein can be extracted, one or more times, by a suitable solvent to produce a crude extract. The solvent for use in preparing the crude extract may be a high-polarity solvent, for example, having a polarity index above 5 and preferably below 7 (e.g., >5.2; >5.5, >5.8, >6 or above and preferably below 7). Examples include, but are not limited to, ethanol, acetone, methanol, water, or a combination thereof. If necessary, the crude extract can be concentrated by a conventional method to produce a concentrated crude extract.

The crude extract can then be brought in contact with a suitable resin (e.g., a non-ionic absorbent resin) under suitable conditions that allow for binding of active components in the crude extract onto the resin. Exemplary resins for use in preparing the Plectranthus amboinicus extract include, but are not limited to, DIAION® HP20, DIAION® HP20SS, Sepabeads® SP207, Amberlite™ XAD-2, or Amberlite™ XAD-4.

Afterwards, the resin can be washed one or more times and eluted with a suitable solvent, for example, a solvent having a polarity index lower than 7, to produce a PA extract, which can then be dried by a conventional method (e.g., freeze-drying, spray-drying, or concentration drying) to produce dried the PA extract, which can be in semisolid or paste form.

In some embodiments, the resin absorption step can be performed by mixing the crude extract with the resin in a container. In other embodiments, the resin separation step can be performed by a chromatography column setting.

Any methods disclosed in US 2023/0000941, which are incorporated by reference herein for the subject matter and purposes referenced herein, can also be used to prepare the PA extract disclosed herein.

### (b) Centella Asiatica (CA) Extract

The Centella asiatica (CA) extract as described herein refers to an extract obtained from whole Centella asiatica plants or a part thereof. The CA extract may comprise asiaticoside.

The CA extract may be present in the topical formation at a concentration of about 0.1-20% (w/w). For example, the topical formulation may comprise about 0.1-0.5%, 0.5-1%, 1-1.5%, 1.5-2%, 2-2.5%, 2.5-3%, 3-3.5%, 3.5-4%, 4-4.5%, 4.5-5%, 5-5.5%, 5.5-6%, 6-6.5%, 6.5-7%, 7-7.5%, 7.5-8%, 8-8.5%, 8.5-9%, 9-9.5%, 9.5-10%, 10-10.5%, 10.5-11%, 11-11.5%, 11.5-12%, 12-12.5%, 12.5-13%, 13-13.5%, 13.5-14%, 14-14.5%, 14.5-15%, 15-15.5%, 15.5-16%, 16-16.5%, 16.5-17%, 17-17.5%, 17.5-18%, 18-18.5%, 18.5-19%, 19-19.5%, or 19.5-20% (w/w) of the CA extract. Alternatively, the topical formulation may comprise about 0.1-5%, 0.1-10%, 0.1-15%, 0.1-20%, 0.5-5%, 0.5-10%, 0.5-15%, or 0.5-20% (w/w) of the CA extract. In specific examples, the topical formulation may comprise about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 percent (w/w) of the CA extract.

The CA extract can be prepared following a conventional method, for example, those described in U.S. Pat. Nos. 5,834,437, 6,417,349, 6,475,536, and 6,267,996, CN 1313124, CN 1089497, and CN 1194154. Below is an example.

CA materials can be prepared via routine practice. Such materials can be fresh CA plant or a part thereof, or dried CA. The CA materials can be extracted with a suitable solvent such as water, ethanol, or a mixture thereof, to produce a crude extract. The crude extract, which can be optionally concentrated, can be either mixed with a suitable resin or loaded onto a column packed with the resin. After being washed for one or more times, the resin can be eluted with a suitable solvent. The resultant eluent can be concentrated to form a paste, which can be dried by a conventional method, for example, vacuum dry, to produce powders of the CA extract. When necessary, the CA powder can be grinded through a mesh (e.g., a No. 100 mesh).

### B. Moisture-Retaining Polymeric Biomaterials

Any of the topical formulations disclosed herein may comprise a moisture-retaining polymeric biomaterial (e.g., collagen as disclosed herein), which may be in an amount ranging from about 0.1% to about 99% (w/w), for example, about 0.1% to about 20%, about 10% to about 50%, about 20% to about 50%, about 30% to about 50%, about 50% to about 70%, or about 70% to about 90%. In some instances, the amount of the moisture-retaining polymeric biomaterial such as collagen in the formulation would depend on the format of the formulation. See examples provided elsewhere herein.

In some embodiments, the moisture-retaining polymeric biomaterial may comprise collagen, karatin, hyaluronate acid or a salt thereof (e.g., sodium hyaluronate), or a combination thereof.

Collagen is the main structural protein in the extracellular matrix in various connective tissues in a human body. Collagen has a triple helix structure formed by elongated fibril. There are multiple forms of collagens, which can be used in the topical formulation provided herein, including Type I, Type II, Type III, Type IV, Type V, or a combination thereof. In some embodiments, the collagen used in the topical formulation provided herein may be from human. Alternatively, the collagen used in the topical formulation may be from a suitable non-human mammal, for example, porcine or bovine. In other examples, the collagen may be from fish. For example, collagen hydrolysate from fish (e.g., having a molecular weight of about MW 600˜1200 Da) may be used. In yet other examples, the collagen may be from bovine or porcine.

Karatin is one of a family of structural fibrous proteins also known as scleroproteins. Alpha-keratin (α-keratin) is a type of keratin found in vertebrates. It is the key structural material making up scales, hair, nails, feathers, horns, claws, hooves, and the outer layer of skin among vertebrates. Keratin also protects epithelial cells from damage or stress.

Hyaluronate acid is an anionic, non-sulfated glycosaminoglycan distributed widely throughout connective, epithelial, and neural tissues. Hyaluronic acid is well known for its ability to hold 1,000 times its weight in water and helps give skin volume and fullness. Sodium hyaluronate is the sodium salt form of hyaluronic acid. It more readily penetrates the deeper layers of your skin, which makes it a great alternative to traditional high molecular weight hyaluronate acid.

### C. Exemplary Forms of Topical Formulations

The topical formulations disclosed herein may be in a suitable format, for example, in the form of a cream, a gel, an ointment, or a sheet dressing.

### (a) Cream Formulation

In some embodiments, the topical formulation described herein may be in the form of a cream. In some instances, the moisture-retaining biomaterial (e.g., collagen as disclosed herein) may constitute about 0.1% to about 20% (w/w) of the cream formulation as disclosed herein. In some examples, the weight percentage of the moisture-retaining biomaterial in the cream formulation may be about 0.5% to about 15%, for example, about 1% to about 10%. In additional examples, the moisture-retaining biomaterial (e.g., collagen as disclosed herein) may constitute about 0.1% to about 5% (e.g., about 0.1% to about 3%, about 0.1% to about 2%, about 0.1% to about 1% or about 0.1% to about 0.5%) by weight in the cream formulation.

In cream formulations, the pharmaceutically effective excipient may comprise an oil base, an emulsifying agent, and an aqueous base (e.g., water). Optionally, the cream formulation described herein may further comprise one or more of the following components: a skin conditioning agent, an emollient, a stiffening agent, a humectant, and a preservative.

The cream formulations disclosed herein possesses superior moisture-retaining capacity with the water loss rate of less than 5%.

### (i) Oil Base

An “oil base” or “oil type ointment” can be any semisolid preparation or vehicle into which an active agent may be incorporated. An oil base may be pharmacologically inert but can entrap water or moisture in order to provide an emollient protective film. In one specific example, the oil base may comprise any petrolatum compound (e.g., petrolatum, white petrolatum, white soft paraffin, liquid petrolatum, liquid paraffin) or any dimethicone compound.

In some embodiments, the oil base may comprise fatty acid ester, aliphatic higher alcohol, paraffin oil, lanolin oil, silicone oil, medium-chain oil, and/or silicone-based polymer.

In some instances, the fatty acid esters may be synthetic or naturally occurring esters of aliphatic carboxylic acids and aliphatic alcohols. Aliphatic carboxylic acids may include saturated or unsaturated aliphatic mono- or di-carboxylic acids, which typically are lower or higher fatty acids having 2 to 24 carbon atoms. In particular, medium or higher fatty acids having 6 to 20 carbon atoms are preferred. By way of non-limiting examples, acetic acid, propionic acid, hexanoic acid, capric acid, caprylic acid, octanoic acid, dioctanoic acid, adipic acid, sebacic acid, palmitic acid, stearic acid, oleic acid, linolic acid, linolenic acid, and/or myristic acid may be used in the cream formulations.

In some instances, the aliphatic higher alcohol may be saturated and unsaturated aliphatic alcohols having 14 to 20 carbon atoms. By way of non-limiting examples, cetanol or palmityl alcohol, stearyl alcohol, oleyl alcohol, and/or hexadecyl alcohol may be used in the cream formulations.

In some instances, the paraffin oil may include those made mainly of the mixture of paraffins having 15 or more carbon atoms. By way of non-limiting examples, fluid paraffin, and/or vaseline (e.g., white vaseline, squalane and squalene) may be used in the cream formulations.

In some instances, the lanolin oil may include lanolin, lanolin wax, hydrogenated or reduced lanolin and its polymer with ethylene oxide.

In some instances, the silicone oil may include admixture of the polymers of dimethylsiloxane. By way of non-limiting examples, dimethylpolysiloxane and its alkyl ester, methylphenylpolysiloxane, and/or glycol methylsiloxane may be used in the cream formulations.

In some instances, the medium-chain oil may have one or more mono-, di-, or triglycerides or combinations thereof. Exemplary glycerides include, but are not limited to, MIGLYOLs®, which are caprylic/capric triglycerides (SASOL Germany GMBH, Hamburg). In some specific examples, MIGLYOLs® include MIGLYOL® 810 (caprylic/capric triglyceride), MIGLYOL® 812 (caprylic/capric triglyceride), MIGLYOL® 816 (caprylic/capric triglyceride), and MIGLYOL® 829 (caprylic/capric/succinic triglyceride). In other examples, the medium-chain oil may comprise other caprylic/capric triglycerides, including, but not limited to, caproic/caprylic/capric/lauric triglycerides, caprylic/capric/linoleic triglycerides, and caprylic/capric/succinic triglycerides. Exemplary caprylic/capric mono-, di-, or triglycerides include, but are not limited to, CAPMUL® MCM, CAPMUL® MCM C10, CAPMUL® MCM C8, CAPMUL® MCM C8 EP, and CAPMUL® 708 G (the CAPMUL® brands are owned by ABITEC, Columbus, Ohio). In still other examples, other mono-, di-, and triglycerides of fractionated vegetable fatty acids, and combinations or derivatives thereof, may be used in the cream formulations disclosed herein. For example, the oil phase may be 1,2,3-propanetriol (e.g., glycerol, glycerin, or glycerine) esters of saturated coconut and palm kernel oil and derivatives thereof.

In some instances, the silicone-based polymer may comprise dimethicone, cyclomethicone, behenocyl dimethicone, dicaprylate/dicaprate dimethicone copolyol, and/or cetyl dimethicone (cety dimethicone).

### (ii) Emulsifying Agent

An “emulsifying agent” is a compound or substance which acts as a stabilizer for a mixture of two or more liquids that are normally immiscible (unmixable or unblendable).

In some embodiments, the emulsifying agent may comprise an anionic surfactant, a cationic surfactant, a zwitterionic surfactant, and/or a non-ionic surfactant.

In some instances, the anionic surfactant may be based on permanent anions (e.g., sulfate, sulfonate, and phosphate) or pH-dependent anions (e.g., carboxylate). Exemplary sulfates include, but are not limited to, alkyl sulfates and alkyl ether sulfates. In some specific examples, alkyl sulfate may be ammonium lauryl sulfate or sodium lauryl sulfate (also known as sodium dodecyl sulfate (SDS)). Alternatively or in addition, alkyl ether sulfates may be sodium laureth sulfate (also known as sodium lauryl ether sulfate (SLES)) or sodium myreth sulfate.

Exemplary sulfonates include, but are not limited to, docusates, sulfonate fluorosurfactants, and alkyl benzene sulfonates. In some specific examples, docusates may be dioctyl sodium sulfosuccinate. Alternatively or in addition, sulfonate fluorosurfactants may be perfluorooctanesulfonate (PFOS) or perfluorobutanesulfonate.

Exemplary phosphates include, but are not limited to, alkyl aryl ether phosphate and alkyl ether phosphate.

In some instances, the cationic surfactant may be based on pH-dependent primary, secondary or tertiary amines. Primary amines become positively charged at pH<10, secondary amines become charged at pH<4, and tertiary amines become charged at pH<9. In one specific example, the cationic surfactant is octenidine dihydrochloride, with a gemini-surfactant structure, derived from pyridine. Octenidine dihydrochloride is active against Gram-positive and Gram-negative bacteria.

Alternatively, the cationic surfactant may be permanently charged quaternary ammonium cation, for example, alkyltrimethylammonium salts. Exemplary alkyltrimethylammonium salts include, but are not limited to, cetyl trimethylammonium bromide (CTAB, also known as hexadecyl trimethyl ammonium bromide), cetyl trimethylammonium chloride (CTAC), cetylpyridinium chloride (CPC), polyethoxylated tallow amine (POEA), benzalkonium chloride (BAC), benzethonium chloride (BZT), 5-Bromo-5-nitro-1,3-dioxane, dimethyldioctadecylammonium chloride, and dioctadecyldimethylammonium bromide (DODAB).

In some instances, the zwitterionic (amphoteric) surfactant may be based on primary, secondary or tertiary amines or quaternary ammonium cation. Exemplary zwitterionic surfactant include, but are not limited to, sulfonate, sultaine, carboxylate, and phosphate. In one specific example, the sulfonate is CHAPS (3-[(3-Cholamidopropyl)dimethylammonio]-1-propanesulfonate). Alternatively or in addition, one specific example of sultaine is cocamidopropyl hydroxysultaine. Still alternatively or in addition, specific examples of carboxylates are amino acids and imino acids. Still alternatively or in addition, one specific example of phosphate is lecithin.

Non-ionic surfactants work by attracting both water and oil at the same time, frequently used as emulsifiers in soaps and cosmetics. In some instances, the non-ionic surfactants include, but are not limited to, alkylamine oxides, mono- and dialkylalkanolamides, fatty acid esters of polyethylenenglycols, ethoxylated fatty acids amides, saturated fatty acid alcohols reacted with ethylene oxide, alkyl polyglycosides, and sorbitan ether esters. In some specific examples, the non-ionic surfactant may be ceteareth-2, ceteareth-3, ceteareth-4, ceteareth-5, ceteareth-6, ceteareth-7, ceteareth-8, ceteareth-9, ceteareth-10, ceteareth-11, ceteareth-12, ceteareth-13, ceteareth-14, ceteareth-15, ceteareth-16, ceteareth-17, ceteareth-18, ceteareth-20, ceteareth-22, ceteareth-23, ceteareth-24, ceteareth-25, ceteareth-27, ceteareth-28, ceteareth-29, ceteareth-30, ceteareth-33, ceteareth-34, ceteareth-40, ceteareth-50, ceteareth-55, ceteareth-60, ceteareth-80, ceteareth-100. Ceteareth-n (where n is a number) refer to polyoxyethylene ethers of a mixture of high molecular mass saturated fatty alcohols, mainly cetyl alcohol (m=15) and stearyl alcohol (m=17). The number n indicates the average number of ethylene oxide residues in the polyoxyethylene chain.

Suitable emulsifying agents for the cream formulations described herein may include, but are not limited to, natural emulsifying agents (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and Veegum [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, propylene glycol monostearate, and polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, and methylcellulose), sorbitan fatty acid esters (e.g., polyoxyethylene sorbitan monolaurate [Tween® 20], polyoxyethylene sorbitan monostearate [Tween® 60], polyoxyethylene sorbitan monooleate [Tween® 80], sorbitan monopalmitate [Span® 40], sorbitan monostearate [Span® 60], sorbitan tristearate [Span® 65], glyceryl monooleate, and sorbitan monooleate [Span® 80]), polyoxyethylene esters (e.g., polyoxyethylene monostearate [Myrj® 45], polyoxyethylene hydrogenated castor oil, polyethoxylated castor oil, polyoxymethylene stearate, and Solutol), sucrose fatty acid esters, polyethylene glycol fatty acid esters (e.g., Cremophor®), polyoxyethylene ethers (e.g., polyoxyethylene lauryl ether [Brij® 30]), and poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, and docusate sodium, and/or combinations thereof. In some specific examples, the emulsifying agent may comprise a polysorbate compound (e.g., a Tween® product), a sorbitan compound (e.g., a Span® product), or a combination thereof.

(iii) Additional Components for Cream Formulations

Optionally, the cream formulations described herein may further comprise one or more skin conditioning agent, one or more emollients, one or more stiffening agents, one or more humectants, one or more preservatives, or a combination thereof.

A “skin conditioning agent” refers to a wide range of substances that help skin look and feel smoother, either immediately or over time. Exemplary skin conditioning agents include, but are not limited to, olive oil, macadamia nut oil, sweet almond oil, grape seed oil, avocado oil, Corn oil, sesame oil, soybean oil, peanut oil, meadowfoam seed oil, safflower seed oil, rosehip (rosa canina) fruit oil, argania spinosa kernel oil, jojoba (Simmondsia chinensis) seed oil, sunflower seed oil, mauritia flexuosa fruit oil, squalane, ethylhexyl palmitate, isopropyl myristate, hydrogenated polyisobutylene, isocetane, isododecane, diethylhexyl carbonate, Dioctyl carbonate, isopropyl lauroyl sarcosinate, isononyl isononanoate, hydrogenated polydecene, triethylhexanoin, cetyl ethylhexanoate, bis-ethoxydiglycol cyclohexane 1,4-dicarboxylate, caprylic/capric triglycerid e), oleyl erucate, octyldodecanol myristate, octyldodecanol, polydimethylsiloxane, octyl polymethylsiloxane, cetyl dimethicone (cety dimethicone), and cyclopentadimethylsiloxane kojic acid, ascorbic acid, ascorbyl glucoside, arbutin, tranexamic acid, nicotinamide, phytosterols, phytosteryl/behenyl/octyldodecyl lauroyl glutamate, phenylethyl resorcinol, turmeric extract, birch bark extract, ceramide 2, ceramide 3, acetyl phytosphingosine, resveratrol, pterocarpus marsupium bark extract, plectranthus barbatus root extract, pepper seed extract, ubiquinone, cholesterol cholesterol), cholesterol stearate, ascorbyl dipalmitate, Tocopherol (vitamin E), tocopheryl acetate, alpha-bisabolol, ascorbyl dipalmitate, ascorbyl tetraisopalmitate, pyridoxine dicaprylate, pyridoxine dipalmitate, retinyl palmitate, phytosteryl/octyldodecyl Lauroyl glutamate (phytosteryl/octyldodecyl lauroyl glutamate), bis-behenyl/isostearyl/phytosteryl dimer dilinoleyl dilinoleate (bis-behenyl/isostearyl/phytosteryl dimer dilinoleyl dilinoleate), phytosteryl macadamie (phytosteryl macadamiate), peptides, and plant extracts.

An “emollient” is a moisturizing agent having the quality of softening or soothing the skin. An emollient is applied directly to the skin to soothe and hydrate it. It covers the skin with a protective film to trap in moisture. Emollients are often used to help manage dry, itchy or scaly skin conditions such as eczema, psoriasis and ichthyosis.

In some instances, suitable emollients include, but are not limited to, PEG 20 almond glycerides, Probutyl DB-10, Glucam™ P-20, Glucam™ E-10, Glucam™ P-10, Glucam™ E-20, Glucam™ P-20 distearate, glycerin, propylene glycol, octoxyglycerin, cetyl acetate, acetylated lanolin alcohol (e.g., Acetulan™), cetyl ether (e.g., PPG-10), myristyril ether (e.g., PPG-3), hydroxylated milk glycerides (e.g., Cremeral HMG), polyquatemium compounds (e.g., U-care compounds), copolymers of dimethyl dialyl ammonium chloride and acrylic acid (e.g., Merquat™), dipropylene glycol methyl ethers (e.g., Dowanol™ DPM, Dow Corning), polypropylene glycol ethers (e.g., Ucon™ 50-HB-600, Union Carbide) and silicon polymers.

In some instances, suitable emollients may include, but are not limited to, hydrocarbon-based emollients (e.g., fatty esters, fatty alcohols, or combinations thereof) include, but are not limited to, diisopropyl adipate, oleyl alcohol, lanolin, isopropyl myristate, isopropyl palmitate, caprylic/capric triglycerides, cetyl lactate, cetyl palmitate, hydrogenated castor oil, glyceryl esters, hydroxycetyl isostearate, hydroxy cetyl phosphate, isopropyl isostearate, isostearyl isostearate, diisopropyl sebacate, polyoxypropylene poloxyethylene cetyl ether (PPG-5-Ceteth-20), 2-ethylhexyl isononoate, 2-ethylhexyl stearate, C12 to C16 fatty alcohol, C12 to C16 fatty alcohol lactate, isopropyl lanolate, 2-ethylhexyl salicylate, and mixtures thereof.

In some instances, one or more emollients may be 1-hexadecanol, acetylated lanolin, behenocyl dimethicone, C12-15 alkyl benzoate, cetearyl octanoate, cocoglycerides, dicaprylate/dicaprate dimethicone copolyol, dimethiconol, dioctyl adipate, glyceryl stearate, isocetyl alcohol, isohexadecane, isopentylcyclohexanone, isopropyl palmitate, lauryl lactate, mineral oil, methoxy PEG-22/dodecyl glycol copolymer, myristyl lactate, ocryldodecyl neopentanoate, octyl cocoate, octyl palmitate, octyl stearate, octyldodecyl neopentanoate, polyglyceryl-4 isosterate, polyoxyl 40 stearate, polyoxymethylene urea, potassium sorbate, propylene glycol, propylene glycol isoceth-3 acetate, and/or propylene glycol myristyl ether acetate.

A “stiffening agent” is a substance that increases the viscosity or stiffness of a preparation. Stiffening agents are used mainly in topical products.

Suitable stiffening agents for the cream formulations described herein include, but are not limited to, cetyl alcohol, stearyl alcohol, cetearyl alcohol, behenyl alcohol, squalyl alcohol, Lauric acid, myristic acid, palmitic acid, stearic acid, beeswax, candelilla wax, carnauba wax), lanolin, ozokerite, jojoba seed wax, paraffin wax, microcrystalline wax, hydrogenated rice bran wax), hydrogenated cocoglycerides, eicosadioate (glyceryl behenate/eicosadioate), myristyl myristate, bis-diglyceryl polyacyladipate-2 2), shea butter (butyrospermum parkii), and palm seed butter (astrocaryum murumuru seed butter).

A “humectant” is a water-absorbing substance used to keep things moist. In pharmaceuticals and cosmetics, humectants can be used in topical forms to increase the solubility of active ingredients, thus increasing the active ingredients' ability to penetrate skin.

In some instances, exemplary humectants suitable for the cream formulations described herein include, but are not limited to, fructose, glucose, glycerin, propylene glycol, glycereth-26, mannitol and urea, pyrrolidone carboxylic acid, hydrolyzed lecithin, coco-betaine, cysteine hydrochloride, glutamine, polyoxypropylene, polyoxyethylene (PPG-15), sodium gluconate, potassium aspartate, oleyl betaine, thiamine hydrochloride, sodium laureth sulfate, sodium hyaluronate, hydrolyzed proteins, hydrolyzed keratin, amino acids, amine oxides, water-soluble derivatives of vitamins A, E and D, amino-functional silicones, ethoxylated glycerin, a-hydroxy acids and salts thereof, and water-soluble fatty oil derivative (e.g., PEG-24 hydrogenated lanolin, almond oil, grape seed oil and castor oil).

Preservatives can be used in the cream formulations (or in other topical formulations disclosed herein) to prevent any microorganism growth. An “antimicrobial preservative” refers to a compound capable of destroying microbes, preventing the multiplication or growth of microbes, or preventing the pathogenic action of microbes. For multiple uses after opening, the topical formulations may contain one or more antimicrobial preservatives to prevent any microorganism growth. For single use packaging, preservatives are optional.

In some instances, exemplary antimicrobial preservatives suitable for the cream formulations described herein include, but are not limited to, silver salts, iodophors, iodine, benzoic acid, dihydroacetic acid, propionic acid, sorbic acid, methyl paraben, ethyl paraben, butyl paraben, cetrimide, benzalkonium chloride, dequalinium chloride, chlorhexidine, chloroeresol, chlorxylenol, benzyl alcohol, bronopol, chlorbutanol, phenoxyethanol, phenylethyl alcohol, 2,4-dichlorobenzyl alcohol, thiomersal, clindamycin, erythromycin, benzoyl peroxide, mupirocin, bacitracin, polymyxin B, neomycin, triclosan, parachlorometaxylene, foscarnet, miconazole, fluconazole, itriconazole, ketoconazole, silver sulfadiazine, octoxyglycerine, biguanides such as, but not limited to, chlorhexidine free base, chlorhexidine palmitate, chlorhexidine diphosphanilate, chlorhexidine digluconate, chlorhexidine diacetate, chlorhexidine dihydrochloride, chlorhexidine dichloride, chlorhexidine dihydroiodide, chlorhexidine diperchlorate, chlorhexidine dinitrate, chlorhexidine sulfate, chlorhexidine sulfite, chlorhexidine thiosulfate, chlorhexidine di-acid phosphate, chlorhexidine difluorophosphate, chlorhexidine diformate, chlorhexidine dipropionate, chlorhexidine di-iodobutyrate, chlorhexidine di-n-valerate, chlorhexidine dicaproate, chlorhexidine malonate, chlorhexidine succinate, chlorhexidine malate, chlorhexidine tartrate, chlorhexidine dimonoglycolate, chlorhexidine monodiglycolate, chlorhexidine dilactate, chlorhexidine di-a-hydroxyisobutyrate, chlorhexidine diglucoheptonate, chlorhexidine di-isothionate, chlorhexidine dibenzoate, chlorhexidine dicinnamate, chlorhexidine dimandelate, chlorhexidine di-isophthalate, chlorhexidine di-2 hydroxynapthoate, chlorhexidine embonate, and parahexamethylenebiguanide (“PHMB”).

### (iv) Exemplary Cream Formulations

In some specific examples, the present disclosure provides cream formulations comprising the PA extract and the CA extract as disclosed herein, collagen as a moisture-retaining polymeric material, and pharmaceutically acceptable excipients comprising (e.g., consisting of) white petroleum and water. In some instances, the weight ratio of the PA extract and the CA extract in the cream formulation ranges from 10:1 to 1:250 (e.g., 10:1, 1:1 or 1:240). Alternatively or in addition, the concentration of the PA extract in the cream formulation may range from 0.1% to 10% (e.g., 0.1% to 5%, 0.1% to 2%, 0.1% to 1%, 1% to 5%, or 5% to 10%) by weight. The concentration of the CA extract in the cream formulation may range from 0.1% to 24% (e.g., 0.1% to 20%, 0.1% to 15%, 0.1% to 10%, 0.1% to 5%, 0.1% to 1%, 1% to 5%, 5% to 10%, 10% to 15%, or 15% to 20%) by weight.

Alternatively or in addition, the cream formulation comprises collagen at an amount of 0.1% to 20% by weight. In some examples, the amount of the collagen may range from 0.1% to 15%, 0.1% to 10%, 0.1% to 5%, or 0.1% to 1%. In other examples, the amount of the collagen may range from 1% to 5%, 5% to 10%, 10% to 15% or 15% to 20%.

### (b) Gel Formulation

The topical formulation described herein may be in the form of a gel. In some instances, the moisture-retaining biomaterial (e.g., collagen as disclosed herein) may constitute about 0.1% to about 70% (w/w) of the gel formulation. In some examples, the weight concentration of the moisture-retaining biomaterial in the gel formulation as disclosed herein may range from about 0.5% to about 70%, for example, about 1% to about 70%, about 10% to about 70%, about 20% to about 70%, about 30% to about 70%, about 40% to about 70%, about 50% to about 70%, or about 60% to about 70%.

In gel formulations, the pharmaceutically effective excipient comprises a gelling agent and an aqueous base (e.g., water). Optionally, the gel formulation described herein may further comprise one or more of the following components: an emollient and a preservative. The gel formulations disclosed herein provide superior stability and touch feeling. Also, the gel formulations can form a membrane over the wound, thus protecting the wound from further harm.

### (i) Gelling Agent

A “gelling agent” is a gel-forming agent that forms a weakly cohesive internal structure when dissolved in a liquid phase as a colloidal mixture. In some embodiments, the gelling agent may comprise a cellulose derivative, a carbomer, or a combination thereof.

In some instances, the gelling agent comprises a cellulose derivative. Exemplary cellulose derivatives include, but are not limited to, cellulose ethers (e.g., carboxymethylcellulose sodium), polysaccharide gums (e.g., xanthan gum), and non-ionic thickeners (e.g., hydroxyethyl cellulose, hydroxymethyl cellulose, and hydroxypropyl cellulose).

In some instances, the gelling agent comprises a carbomer, which is a crosslinked or non-crosslinked poly(acrylic acid) polymer or co-polymer. Exemplary carbomers include, but are not limited to, CARBOPOL® products (e.g., CARBOPOL® 934, CARBOPOL® 940, CARBOPOL® 941, CARBOPOL® 980, CARBOPOL® 981, CARBOPOL® 1342, and CARBOPOL® 1382).

In one specific example, the gelling agent comprises carboxymethyl cellulose.

### (ii) Additional Components for Gel Formulations

Optionally, the gel formulations described herein may further comprise one or more emollients, one or more preservatives, or a combination thereof.

Any of the emollients disclosed herein may be used in preparing the gel formulation. In some instances, exemplary emollients suitable for the gel formulations described herein include, but are not limited to, glycerine, glyceryl oleate, caprylyl glycol, triglycerides (e.g., caprylic/capric triglyceride), silicone oils (e.g., cyclomethicone), ester oils (e.g., butyl myristate, isopropyl myristate, cetyl myristate, isopropyl palmitate, isopropyl stearate, octyl stearate, isocearyl stearate), organic fatty alcohols (e.g., oleic alcohol, linolenic alcohol, linoleic alcohol, isostearyl alcohol, octyl dodecanol), long chain fatty acids, liquid water-soluble polyols, propylene glycol, sorbitol, polyethylene glycol, ethoxylated/propoxylated ethers of methyl glucose (e.g., methyl gluceth-20) and ethoxylated/-propoxylated ethers of lanolin alcohol (e.g., Solulan-75), coco and tallow fatty acids, nonoclusive liquid water soluble polyols, essential amino acids, and sodium pyrrolidone carboxylic acid (or sodium dl-pyrrolidone carboxylate), urea, L-proline, guanidine and pyrrolidone; hexadecyl, myristyl, isodecyl or isopropyl esters of adipic, lactic, oleic, stearic, isostearic, myristic or linoleic acids and corresponding alcohol esters such as sodium isostearoyl-2 lactylate, sodium capryl lactylate, aloe vera gel and acetamide MEA.

Any of the preservatives suitable for topical formulations (e.g., those disclosed herein) may be used herein.

### (c) Ointment Formulation

The topical formulation described herein may be in the form of an ointment. In some instances, the moisture-retaining biomaterial (e.g., collagen as disclosed herein) may constitute about 0.1% to about 70% (w/w) of the ointment formulation. In some embodiments, the weight percentage of the moisture-retaining biomaterial in the ointment formulation as disclosed herein may range from about 1% to about 10%, about 10% to about 20%, about 20% to about 30%, about 30% to about 40%, about 40% to about 50%, about 50% to about 60% or about 60% to about 70% by weight. In some examples, the weight percentage of the moisture-retaining biomaterial in the ointment formulation as disclosed herein may range from about 0.5% to about 20%, about 1% to about 15%, or about 5% to about 10%.

In ointment formulations, the pharmaceutically effective excipient comprises an ointment base. Optionally, the ointment formulation described herein may further comprise one or more preservatives. The ointment formulations disclosed herein provide superior stability and moisture-retaining capacity with the water loss rate of less than 1%.

### (i) Ointment Base

An “ointment base” can be any semisolid preparation or vehicle into which an active agent may be incorporated. Exemplary ointment bases include, but are not limited to, oleaginous ointment bases (e.g., white petrolatum or white ointment), absorption ointment bases (e.g., hydrophilic petrolatum, anhydrous lanolin, Aquabase™, Aquaphor®, and Polysorb®), water/oil emulsion ointment bases (e.g., cold cream, hydrous lanolin, rose water ointment, Hydrocream™, Eucerin®, and Nivea®), oil/water emulsion ointment bases (e.g., hydrophilic ointments, Dermabase™, Velvachol®, and Unibase®), and water-miscible ointment bases (e.g., polyethylene glycol (PEG) ointment and Polybase™). Ointment bases may be pharmacologically inert but can entrap water in order to provide an emollient protective film. In a specific embodiment, the ointment base may be any petrolatum compound (e.g., petrolatum, white petrolatum, white soft paraffin, liquid petrolatum, liquid paraffin). In a further specific embodiment, the ointment base is white petrolatum (CAS number 8009-03-8).

There are two types of ointment that can be used in the ointment formulations described herein. In some embodiments, the ointment base may be PEG type ointment. Such ointment base comprises a polyethylene glycol. Exemplary PEG type ointments include, but are not limited to, PEG 600, PEG 400, PEG 300, PEG 100, PEG 800, PEG 1000, PEG 3350, PEG 3450 PEG 4000, PEG-stearate, PEG40-stearate, PEG-Laureate, lecithin, phosphatidylcholine, PEG-oleate, and PEG-glycerol.

Alternatively or in addition, the ointment base may be oil type ointment. In some embodiments, the oil type ointment may comprise fatty acid ester, aliphatic higher alcohol, paraffin oil, lanolin oil, silicone oil, medium-chain oil, and/or silicone-based polymer.

In some instances, the fatty acid esters may be synthetic or naturally occurring esters of aliphatic carboxylic acids and aliphatic alcohols. Aliphatic carboxylic acids may include saturated or unsaturated aliphatic mono- or di-carboxylic acids, which typically are lower or higher fatty acids having 2 to 24 carbon atoms. In particular, medium or higher fatty acids having 6 to 20 carbon atoms are preferred. By way of non-limiting examples, acetic acid, propionic acid, hexanoic acid, capric acid, caprylic acid, octanoic acid, dioctanoic acid, adipic acid, sebacic acid, palmitic acid, stearic acid, oleic acid, linolic acid, linolenic acid, and/or myristic acid may be used in the cream formulations.

In some instances, the aliphatic higher alcohol may be saturated and unsaturated aliphatic alcohols having 14 to 20 carbon atoms. By way of non-limiting examples, cetanol or palmityl alcohol, stearyl alcohol, oleyl alcohol, and/or hexadecyl alcohol may be used in the cream formulations.

In some instances, the paraffin oil may include those made mainly of the mixture of paraffins having 15 or more carbon atoms. By way of non-limiting examples, fluid paraffin, and/or vaseline (e.g., white vaseline, squalane and squalene) may be used in the cream formulations.

In some instances, the lanolin oil may include lanolin, lanolin wax, hydrogenated or reduced lanolin and its polymer with ethylene oxide.

In some instances, the silicone oil may include admixture of the polymers of dimethylsiloxane. By way of non-limiting examples, dimethylpolysiloxane and its alkyl ester, methylphenylpolysiloxane, and/or glycol methylsiloxane may be used in the cream formulations.

In some instances, the medium-chain oil may have one or more mono-, di-, or triglycerides or combinations thereof. Exemplary glycerides include, but are not limited to, MIGLYOLs®, which are caprylic/capric triglycerides (SASOL Germany GMBH, Hamburg). In some specific examples, MIGLYOLs® include MIGLYOL® 810 (caprylic/capric triglyceride), MIGLYOL® 812 (caprylic/capric triglyceride), MIGLYOL® 816 (caprylic/capric triglyceride), and MIGLYOL® 829 (caprylic/capric/succinic triglyceride). In other examples, the medium-chain oil may comprise other caprylic/capric triglycerides, including, but not limited to, caproic/caprylic/capric/lauric triglycerides, caprylic/capric/linoleic triglycerides, and caprylic/capric/succinic triglycerides. Exemplary caprylic/capric mono-, di-, or triglycerides include, but are not limited to, CAPMUL® MCM, CAPMUL® MCM C10, CAPMUL® MCM C8, CAPMUL® MCM C8 EP, and CAPMUL® 708 G (the CAPMUL® brands are owned by ABITEC, Columbus, Ohio). In still other examples, other mono-, di-, and triglycerides of fractionated vegetable fatty acids, and combinations or derivatives thereof, may be used in the cream formulations disclosed herein. For example, the oil phase may be 1,2,3-propanetriol (glycerol, glycerin, glycerine) esters of saturated coconut and palm kernel oil and derivatives thereof.

In some instances, the silicone-based polymer may comprise dimethicone, cyclomethicone, behenocyl dimethicone, dicaprylate/dicaprate dimethicone copolyol, and/or cetyl dimethicone (cety dimethicone).

### (ii) Additional Components for Ointment Formulations

Optionally, the ointment formulations described herein may further comprise one or more preservatives. Any of the preservatives suitable for topical formulations (e.g., those disclosed herein) may be used herein.

(iii) Exemplary Ointment Formulations

In some specific examples, the present disclosure provides ointment formulations comprising the PA extract and the CA extract as disclosed herein, collagen as a moisture-retaining polymeric material, and pharmaceutically acceptable excipients comprising (e.g., consisting of) white petroleum. Such ointment formulations may be free of water. In some instances, the weight ratio of the PA extract and the CA extract in the cream formulation ranges from 1:1 to 1:400 (e.g., 1:1, 1:10, 1:50, 1:100, 1:200, 1:300, or 1:400). Alternatively or in addition, the concentration of the PA extract in the cream formulation may range from 0.1% to 1% (e.g., 0.1% to 0.5%) by weight. The concentration of the CA extract in the cream formulation may range from 0.1% to 40% (e.g., 0.1% to 30%, 0.1% to 20%, 0.1% to 15%, 0.1% to 10%, 0.1% to 5%, 0.1% to 1%, 1% to 5%, 5% to 10%, 10% to 15%, or 15% to 20%, 20% to 30%, or 30% to 40%) by weight.

Alternatively or in addition, the cream formulation comprises collagen at an amount of 0.1% to 70% by weight. In some examples, the amount of the collagen may range from 1% to 70%, 5-60%, or 10-50%).

### (d) Sheet Dressing

The topical formulation described herein may be in the form of a sheet dressing. In some instances, the moisture-retaining biomaterial (e.g., collagen as disclosed herein) may constitute about 25% to about 90% (w/w) of the sheet dressing formulation. In some examples, the weight percentage of the moisture-retaining biomaterial (e.g., collagen) in any of the sheet dressings disclosed herein may range from about 30% to about 70%, about 40% to about 60%, about 25% to about 50%, or about 50% to about 70%). In other examples, the weight percentage of the moisture-retaining biomaterial (e.g., collagen) in any of the sheet dressings disclosed herein may range from about 80% to about 90%. In some instances, the weight percentage of the moisture-retaining biomaterial (e.g., collagen) in any of the sheet dressings disclosed herein does not exceed 95%. In some instances, the weight percentage of the moisture-retaining biomaterial (e.g., collagen) in any of the sheet dressings disclosed herein does not exceed 90%.

In sheet dressing formulations, the pharmaceutically effective excipient comprises a dressing. Optionally, the sheet dressing formulation described herein may further comprise one or more of the following components: an antioxidant, a growth factor, and/or an extracellular matrix. The sheet dressing formulations disclosed herein are sterile and can maintain long-term efficacy, e.g., the dressing can stay on the wound and remain effective for longer than 4 days.

### (i) Dressing

A “dressing” or a “sheet dressing” is a sterile pad applied to a wound to promote healing and protect the wound from further harm. A dressing is designed to be in direct contact with the wound.

In some embodiments, the dressing suitable for the topical formulation described herein may be a bandage, a membrane, a synthetic or biological (e.g., acellular or cellular) hydrogel, a hydrocolloid, a film, a foam, a gauze, a dermal patch, an adhesive tape, a skin substitute, a paste, a powder, or a fiber (e.g., an anionic, amphoteric or hydrophilic polymer).

### (ii) Additional Components for Sheet Dressing

In some embodiments, the dressing formulation disclosed herein may further comprise one or more antioxidant, one or more growth factors, one or more extracellular matrix, or a combination thereof.

An “antioxidant” is a compound that inhibits oxidation (usually occurring as autoxidation), a chemical reaction that can produce free radicals. Autoxidation leads to degradation of organic compounds, including living matter. Antioxidants can be added to topical formulations to extend their usable lifetimes.

In some embodiments, the antioxidants suitable for the sheet dressing may comprise Vitamin A and other carotenoid compounds, vitamin E and other tocopherols, and proanthocyanodins and their derivatives.

In some embodiments, the antioxidants may comprise sulfites. Suitable sulfites include, but are not limited to, sodium bisulfite (a mixture of monosodium sulfite (NaHSO₃) and sodium metabisulfite (Na₂S₂O₅)), sodium metabisulfite, potassium metabisulfite (K₂SO₅), sulfur dioxide, and acids. In some specific examples, the acids may be ascorbic and benzoic acids.

Growth factors can bind to ECM proteins. The binding of growth factors to the ECM is a major mechanism regulating growth factor activity.

In some embodiments, exemplary growth factors that bind to ECM proteins and are suitable for the sheet dressing formulations described herein include, but are not limited to, platelet-derived growth factor (PDGF), transforming growth factor-β (TGF-β), fibroblast growth factor (FGF), insulin-like growth factor (IGF), and epidermal growth factor (EGF). Exemplary ECM proteins include, but are not limited to, keratin, fibronectin, and vitronectin.

(iii) Exemplary Sheet Dressing

In some specific examples, the present disclosure provides sheet dressing formulations comprising the PA extract and the CA extract as disclosed herein and collagen as a moisture-retaining polymeric material. In some instances, such sheet dressing formulations may be free of other components such as pharmaceutically acceptable excipients as those disclosed herein. In some instances, the weight ratio of the PA extract and the CA extract in the cream formulation ranges from 1:2 to 1:10 (e.g., 1:2, 1:4, or 1:5). Alternatively or in addition, the concentration of the PA extract in the cream formulation may range from 1% to 5% (e.g., 2%-4%) by weight. The concentration of the CA extract in the cream formulation may range from 5% to 20% (e.g., 5% to 18% or 5% to 16%) by weight.

Alternatively or in addition, the cream formulation comprises collagen at an amount of 70%-90% by weight. In some examples, the amount of the collagen may range from 70% to 80%, 80-85%, or 85-90%).

### II. Methods for Promoting Wound Healing

Any of the topical formulations described herein can be used for promoting wound healing in a subject in need of the treatment. The topical formulation may be applied to a wound site following a suitable dosage and treatment regimen. The dosage and administration regimen for the described method will depend on the nature and condition of the wound being treated, the age and condition of the patient, and any prior or concurrent therapy. In some instances, the topical formulation can be applied once every week, once every other day, once daily, twice daily, three times daily, or four time daily for a suitable period of time. The treatment may be terminated when the wound is recovered. When necessary, the treatment may resume, for example, if the wound recurs.

The term “wound” refers to an injury to living tissue caused by a cut, blow, or other impact (e.g., caused by a medical condition such as a skin disorder), typically one in which the skin is cut or broken. Wound may be associated with a medical condition, for example, a skin disorder. The term “wound healing” denotes the dynamic and complex process of replacing devitalized or missing cellular structures and/or tissue layers. The term “promotion of wound healing” or “promoting wound healing” denotes the inducement of an increased level or rate of replacement for devitalized or missing cellular structures and/or tissue layers. As an example, promotion of wound healing may be indicated by partial or complete ulcer closure or an increase in the healing rate of an ulcer (including but not limited to more rapid changes in ulcer size, area, or severity, a more rapid closure of the ulcer, and/or an increase in the percentage change from baseline in ulcer size, area, or severity when compared to a control ulcer treated with a placebo).

The subject to be treated by the topical formulation described herein can be a human or a non-human mammal. In some embodiments, the subject is a human patient having an open or acute wound, which refers to an injury or damage to living tissues (e.g., skin) that cause a disruption in the normal continuity of biological structures. An open wound may include, but is not limited to, an abrasion, incision, laceration, puncture, avulsion, cut, or other similar injuries.

In other embodiments, the subject is a human patient having a chronic wound, which can be injuries or damage to living tissues (e.g., skin) that cause a disruption in the normal continuity of biological structures and do not heal in an orderly set of stages and/or in a predictable amount of time. A chronic wound may include, but is not limited to, a surgical wound, a traumatic wound, a pressure ulcer, a venous ulcer, or a diabetic ulcer. In other examples, a chronic wound may be associated with a disease or disorder, for example, a carcinoma, burn, bedsore, a skin disorder such as atopic dermatitis.

In some embodiments, the subject to be treated by the methods described herein suffers from a severe wound, for example, having an ulcer with an area greater than 2 cm² (e.g., 3 cm², 4 cm² or 5 cm²). In some examples, the subject suffers from one or more plantar ulcers.

### III. Kits for Use in Promoting Wound Healing

The present disclosure also provides kits for use in promoting wound healing. Such kits may include one or more containers comprising a topical formulation as described herein, which comprises a PA extract, a CA extract, a moisture-retaining polymeric biomaterial, and a pharmaceutically effective excipient.

In some embodiments, the kit may comprise instructions for use in accordance with any of the methods described herein. The included instructions may comprise a description of administration of the topical formulation to promote wound healing according to any of the methods described herein. The kit may further comprise a description of selecting an individual suitable for treatment based on identifying whether that individual has wounds in need of treatment.

The instructions relating to the use of a topical formulation generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for promoting wound healing. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like.

Kits may optionally provide additional components such as interpretive information. Normally, the kit comprises a container and a label or package insert(s) on or associated with the container. In some embodiments, the invention provides articles of manufacture comprising contents of the kits described above.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Water Retention Capacity Test

A moist environment has been proven to facilitate the healing process of a wound as it prevents dehydration, enhances angiogenesis and collagen synthesis, and increases breakdown of dead tissue and fibrin. The topical formulation described herein helps to maintain the wound moist environment.

Water retention capacity testing was conducted on the topical formulations. The testing utilized the Automatic Film Applicator Standard (TQC SHEEN, AB4220) for film preparation. The test article was spread on the film which sealed to the edge of the cup. The water retention capacity test was determined by placing the cup at the stability chamber conditioning at temperate 32±1° C., 50±2% relative humidity for 21 hours. The total water loss rate was calculated.

For the five cream formulations that were tested, water and white petrolatum were used as the excipients. For the three ointment formulations that were tested, white petrolatum was used as the excipient.

The results show that the combination of PA extract and CA extract provided good water retention capacity of 96.2%. The combination of PA extract, CA extract, and collagen provided exceptional water retention capacity of from 97.1% to 100.0%. Such water retention rates are superior to the commercial skin conditioning products such as ALHYDRAN® (94.5%), BIAFINE® (93.8%) and Life Fusion® (93.1%). See, Table 1.

In addition, three sheet formulations containing the PA extract, the CA extract, and collagen as indicated in Table 1 were also examined for water retention capabilities. Two tested sheet formulations showed water retention rates superior to that of the commercial collagen sheet product Life Fusion® (93.1%).

TABLE 1
Water Retention Capacity Test

| | | | | Water | 21 hours |
|---|---|---|---|---|---|
| Topical Formulation (Components) | | | | Loss | Water |
| Formulation | PA extract | CA extract | Collagen | Rate | Retention |
| type | (A)% (w/w) | (B)% (w/w) | (C)% (w/w) | (%) | Rate (%) |
| Cream | 1 | 0.25 | 0 | 3.8 | 96.2 |
| | 0.1 | 0.1 | 0.1 | 2.9 | 97.1 |
| | 10 | 0.1 | 0.1 | 0.9 | 99.1 |
| | 0.1 | 24 | 0.1 | 1.6 | 98.4 |
| | 0.1 | 0.1 | 20 | 2.7 | 97.3 |
| Ointment | 20 | 0.1 | 0.1 | 0.0 | 100 |
| | 0.1 | 40 | 0.1 | 0.0 | 100 |
| | 0.1 | 0.1 | 70 | 0.0 | 100 |
| Sheet | 3.8 | 15.4 | 80.8 | 4.2 | 95.8 |
| | 2 | 8 | 89.8 | 6.6 | 93.4 |
| | 0.9 | 3.7 | 95.4 | 8.0 | 92.0 |

| | | |
|---|---|---|
| Control 1 (ALHYDRAN ®, cream formulation with no | 5.5 | 94.5 |
| collagen) | | |
| Skin/wound Conditioning Formulation (active components): | | |
| Aloe Vera extract | | |
| Control 2 (BIAFINE ®; cream formulation with no collagen) | 6.2 | 93.8 |
| Skin/wound Conditioning Formulation (active components): | | |
| CA extract | | |
| Control 3 (Life Fusion ®; collagen sheet wound dressing) | 6.9 | 93.1 |
| Skin/wound Conditioning Formulation (active components): | | |
| Collagen (>99% by weight) | | |

### Other Embodiments

All of the features disclosed in this specification may be combined together in any manner. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents and published patent applications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A topical formulation, comprising:
(i) a Plectranthus amboinicus (PA) extract;
(ii) a Centella asiatica (CA) extract;
(iii) a moisture-retaining polymeric biomaterial.

2. The topical formulation of claim 1, further comprises (iv) a pharmaceutically effective excipient.

3. The topical formulation of claim 1, wherein in the topical formulation:
(i) the amount of the PA extract is about 0.1-20% (w/w);
(ii) the amount of the CA extract is about 0.1-40% (w/w); and/or
(iii) the amount of the moisture-retaining polymeric biomaterial is about 0.1-90% (w/w); optionally about 0.1% to about 70% (w/w).

4. The topical formulation of claim 1, wherein the moisture-retaining polymeric biomaterial comprises collagen, karatin, hyaluronate acid or a salt thereof, which optionally is sodium hyaluronate, or a combination thereof.

5. The topical formulation of claim 4, wherein the moisture-retaining polymeric biomaterial comprises collagen from fish, porcine, or bovine.

6. The topical formulation of claim 1, wherein the topical formulation is in the form of a cream, a gel, an ointment, or a sheet dressing.

7. The topical formulation of claim 6, wherein the topical formulation is a cream formulation, and wherein topical formulation comprises a pharmaceutically effective excipient, which comprises an oil base, an emulsifying agent, and water.

8. The topical formulation of claim 7, wherein the oil base comprises fatty acid ester, aliphatic higher alcohol, paraffin oil, lanolin oil, silicone oil, medium-chain oil, and/or silicone-based polymer.

9. The topical formulation of claim 8, wherein the oil base comprises petrolatum, dimethicone, or a combination thereof.

10. The topical formulation of claim 7, wherein the emulsifying agent comprises an anionic surfactant, a cationic surfactant, a zwitterionic surfactant, and/or a non-ionic surfactant.

11. The topical formulation of claim 10, wherein the emulsifying agent comprises a polysorbate compound, a sorbitan compound, or a combination thereof.

12. The topical formulation of claim 7, wherein the pharmaceutically effective excipient further comprises one or more of the following:
(a) a skin conditioning agent;
(b) an emollient;
(c) a stiffening agent;
(d) a humectant; and
(e) a preservative.

13. The topical formulation of claim 7, wherein the topical formulation comprises the PA extract in an amount of about 0.1-10% by weight, the CA extract in an amount of about 0.1-24% by weight; and/or the collagen in an amount of about 0.1-20% by weight.

14. The topical formulation of claim 13, wherein the topical formulation comprises the pharmaceutically effective excipient, which comprises white petrolatum and water.

15. The topical formulation of claim 6, wherein the topical formulation is a gel formulation, and wherein the pharmaceutically effective excipient comprises a gelling agent and water.

16. The topical formulation of claim 15, wherein the gelling agent comprises a cellulose derivative, a carbomer, or a combination thereof.

17. The topical formulation of claim 16, wherein the gelling agent comprises carboxymethyl cellulose.

18. The topical formulation of claim 15, wherein the pharmaceutically effective excipient further comprises an emollient and/or a preservative.

19. The topical formulation of claim 6, wherein the topical formulation is an ointment formulation, and wherein the pharmaceutically effective excipient comprises an ointment base.

20. The topical formulation of claim 19, wherein the ointment base comprises a polyethylene glycol, an oil base, or a combination thereof.

21. The topical formulation of claim 20, wherein the ointment base comprises petrolatum, dimethicone, or a combination thereof.

22. The topical formulation of claim 19, wherein the pharmaceutically effective excipient further comprises a preservative.

23. The topical formulation of claim 19, wherein the topical formulation comprises the PA extract in an amount of about 0.1-20% by weight; the CA extract in an amount of about 0.1-40% by weight; and/or the collagen in an amount of about 0.1-70% by weight.

24. The topical formulation of claim 23, wherein the pharmaceutically acceptable excipient therein comprises white petrolatum.

25. The topical formulation of claim 6, wherein the topical formulation is a sheet dressing, and wherein the pharmaceutically effective excipient comprises a dressing.

26. The topical formulation of claim 25, wherein the dressing is a bandage, a membrane, a synthetic or biological hydrogel, a hydrocolloid, a film, a foam, a gauze, a dermal patch, an adhesive tape, a skin substitute, a paste, a powder, or a fiber.

27. The topical formulation of claim 25, wherein the pharmaceutically effective excipient further comprises an antioxidant, a growth factor, and/or an extracellular matrix protein.

28. The topical formulation of claim 25, wherein the topical formulation comprises the PA extract in an amount of about 2-4% by weight, the CA extract in an amount of about 8-15% by weight, and/or collagen in an amount of about 80-90% by weight.

29. A method for promoting wound healing in a subject, the method comprising placing the topical formulation of claim 1 on a wound site of a subject in need thereof.

30. The method of claim 29, wherein the topical formulation is placed on the wound site at least once per day.

31. The method of claim 29, wherein the subject is a human patient having an acute or a chronic wound.
